(19) United States
(12) Patent Application Publication
(10) Pub. No.: US 2019/0223841 A1
(43) Pub. Date: Jul. 25, 2019

(54) CONTROL DEVICE, CONTROL METHOD, CONTROL SYSTEM, AND NON-TRANSITORY COMPUTER-READABLE MEDIUM

(71) Applicant: CANON KABUSHIKI KAISHA, Tokyo (JP)

(72) Inventors: Nobu Miyazawa, Yokohama-shi (JP); Taku Inoue, Machida-shi (JP)

(21) Appl. No.: 16/374,595

(22) Filed: Apr. 3, 2019

Related U.S. Application Data
(63) Continuation of application No. PCT/JP2017/036020, filed on Oct. 3, 2017.

(30) Foreign Application Priority Data
Oct. 7, 2016 (JP) 2016-198892

(51) Int. Cl. A61B 8/00; A61B 8/14; A61B 5/00; A61B 8/08; G16H 30/40

(52) U.S. Cl. CPC A61B 8/54; A61B 8/463; A61B 8/14; G16H 30/40; A61B 8/467; A61B 8/5261; A61B 8/4483; A61B 5/0095

(57) ABSTRACT

An imaging error instruction regarding a first medical image is accepted, a second medical image that is different from the first medical is selected based on information attached to the first medical image, and the first medical image and the second medical image are taken to be imaging errors.

### CROSS-REFERENCE TO RELATED APPLICATIONS

This application is a Continuation of International Patent Application No. PCT/JP2017/036020, filed Oct. 3, 2017, which claims the benefit of Japanese Patent Application No. 2016-198892, filed Oct. 7, 2016, both of which are hereby incorporated by reference herein in their entirety.

### TECHNICAL FIELD

The present invention relates to a control device, a control method, a control system, and a program.

### BACKGROUND ART

Information systems for acquiring and managing medical images are in use. In such information systems, there are cases where a user checks an acquired medical image, for example, and determines whether or not taking of that medical image has failed (imaging error). PTL 1 discloses determining whether or not taking of the medical image has failed each time a medical image is taken.

### CITATION LIST

### Patent Literature

PTL 1 Japanese Patent Laid-Open No. 2002-159483

### SUMMARY OF INVENTION

In a case where multiple related medical images are acquired, determining whether or not taking each of these medical image has failed, without taking the relation thereof into consideration, could be a troublesome process.

A control device according to an embodiment of the present invention includes: accepting means configured to accept imaging error instructions regarding a first medical image acquired based on signals of ultrasound waves from an object; selecting means configured to select a second medical image that is a different medical image from the first medical image and that is acquired based on signals of ultrasound waves from the object, based on information attached to the first medical image; and processing means configured to attach, to the first medical image and the selected second medical image, information indicating that the medical images are imaging errors.

Further features of the present invention will become apparent from the following description of exemplary embodiments with reference to the attached drawings.

### BRIEF DESCRIPTION OF DRAWINGS

FIG. 1 is a diagram illustrating an example of the functional configuration of a control device according to an embodiment of the present invention.

FIG. 2 is a diagram illustrating an example of the hardware configuration of the control device according to the embodiment of the present invention.

FIG. 3 is a flowchart illustrating an example of processing performed by the control device according to the embodiment of the present invention.

FIGS. 4A and 4B are a flowchart illustrating an example of processing performed by the control device according to the embodiment of the present invention.

FIG. 5 is a flowchart illustrating an example of processing performed by the control device according to the embodiment of the present invention.

FIG. 6 is a diagram illustrating an example of a screen displayed by the control device according to the embodiment of the present invention.

FIG. 7A is a diagram illustrating an example of a screen displayed by the control device according to the embodiment of the present invention.

FIG. 7B is a diagram illustrating an example of a screen displayed by the control device according to the embodiment of the present invention.

FIG. 8 is a diagram illustrating an example of a screen displayed by the control device according to the embodiment of the present invention.

FIG. 9A is a diagram illustrating an example of a screen displayed by the control device according to the embodiment of the present invention.

FIG. 9B is a diagram illustrating an example of a screen displayed by the control device according to the embodiment of the present invention.

FIG. 10 is a diagram illustrating an example of the configuration of a system including the control device according to the embodiment of the present invention.

### DESCRIPTION OF EMBODIMENTS

An embodiment of the present invention will be described below with reference to the drawings. In the present specifications, acoustic waves generated by expansion occurring in the body of a subject of examination due to the object being irradiated by light are referred to as photoacoustic waves. Also, acoustic waves transmitted from a transducer, and reflected waves (echo) where the transmitted acoustic waves are reflected in the body of the subject of examination, are referred to as ultrasound waves.

Imaging methods using ultrasound and techniques of imaging using photoacoustic waves are being used as methods for minimally-invasive imaging of states inside the body of a subject of examination. The method of imaging using ultrasound is a method where ultrasound waves emitted from a transducer, for example, are reflected at tissues inside the body of the subject due to difference in acoustic impedance, and an image is generated based on the time for reflected waves to arrive at the transducer, and the intensity of the reflective waves. An image made by imaging using ultrasound waves will be referred to hereinafter as an ultrasound image. The user operates a probe while changing the angle and so forth, and can observe ultrasound images of various types of cross-sections in real-time. Ultrasound images visualize shapes of organs and tissue, and is being used to discover tumors and so forth. The method of imaging using photoacoustic waves is a method where an image is generated based on photoacoustic waves generated by thermal expansion of tissue within the body of the subject that has been irradiated by light, for example. An image where imaging has been performed using photoacoustic waves will be referred to hereafter as a photoacoustic image. Photoacoustic images visualize information relating to optical properties, such as the degree of absorption of light by the tissues. It is known that blood vessels can be visualized by photoacoustic images due to optical properties of hemoglobin, for example, and studies are being made for use in evaluation of malignancy of tumors or the like.

There are cases where various types of information are collected by imaging different phenomena at the same part of the body of a subject based on different principles, in order to improve the accuracy of diagnosis. For example, there are cases where diagnosis regarding cancer is performed by combining form information obtained by computed tomography (CT) images and functional information related to metabolism obtained from positron-emission tomography (PET) images. It is thought that performing diagnosis using information obtained by imaging different phenomena based on different principles is effective in improving accuracy of diagnosis.

Imaging devices for acquiring images that combine the properties of the above-described ultrasound images and photoacoustic images are being studied. Particularly, both ultrasound images and photoacoustic images are imaged using ultrasound waves from the body of the subject, so imaging of ultrasound images and photoacoustic images can be performed using the same imaging device. More specifically, reflected waves that the object has been irradiated by and photoacoustic waves can be received at the same transducer. Accordingly, ultrasound wave signals and photoacoustic signals can be acquired at a single probe, so an imaging device that performs imaging of ultrasound images and imaging of photoacoustic images can be realized with the same device, without the hardware configuration becoming complicated.

For example, in a case of performing examination using an imaging device that performs imaging of ultrasound images and imaging of photoacoustic images as described above, it is envisioned that various images will be generated during a single examination. An example of a case will be described where imaging of ultrasound images is being performed, and in conjunction with this, imaging of photoacoustic images is performed by the user performing input of instructions instructing irradiation by light as appropriate. A moving image made up of a group of a series of ultrasound images, or ultrasound images at timings instructed by the user, are generated in a single examination. Each time the body of the subject is irradiated by light in a single examination, photoacoustic waves are generated in the body, and various types of photoacoustic images are generated which will be described later. In a case where irradiation by light is performed multiple times in a single examination, multiple photoacoustic images can be generated at each timing of irradiation by light.

Thus, a series of ultrasound images is generated for a part extracted in accordance with a series of probe operations, in one examination. Further, multiple photoacoustic images can be generated from photoacoustic signals acquired in accordance with irradiation by light. photoacoustic signals acquired in accordance with irradiation by light, and ultrasound signals acquired in the same time or a near to this irradiation by light can be thought to be photoacoustic waves and returning waves from approximately the same part in the object. Accordingly, a medical image generated from these photoacoustic signals and ultrasound signals can be thought to have been taken at a generally the same timing, and to be imaging approximately the same port. An ultrasound image, and a photoacoustic image taken at generally the same timing as the ultrasound image are preferably stored in a correlated manner for comparison in interpreting at a later time.

In a case where the user checks an image that has been taken, there are cases where the user notices that the image is not suitable for interpreting, i.e., the image is an imaging error image (hereinafter may be referred to simply as “imaging error”). If the image is blurred due to body movement of the patient, or conditions regarding imaging are inappropriate, the image that has been taken is considered to be an imaging error image. The user that has recognized the checked image to be an imaging error image gives an instruction that this image is an imaging error image. In a case where there are images taken at generally the same time as the image that is considered to be an imaging error image, there is a chance that these images also are imaging error images. In a case where the user does not notice that there are images taken at generally the same timing, images that actually should be considered to be imaging error images may be used for interpretation. Even if the user does recognize that there are images taken at generally the same timing, operations of instructing that all of these are imaging error images may be troublesome work for the user. The embodiment according to the present invention is proposed in light of such problems, for example.

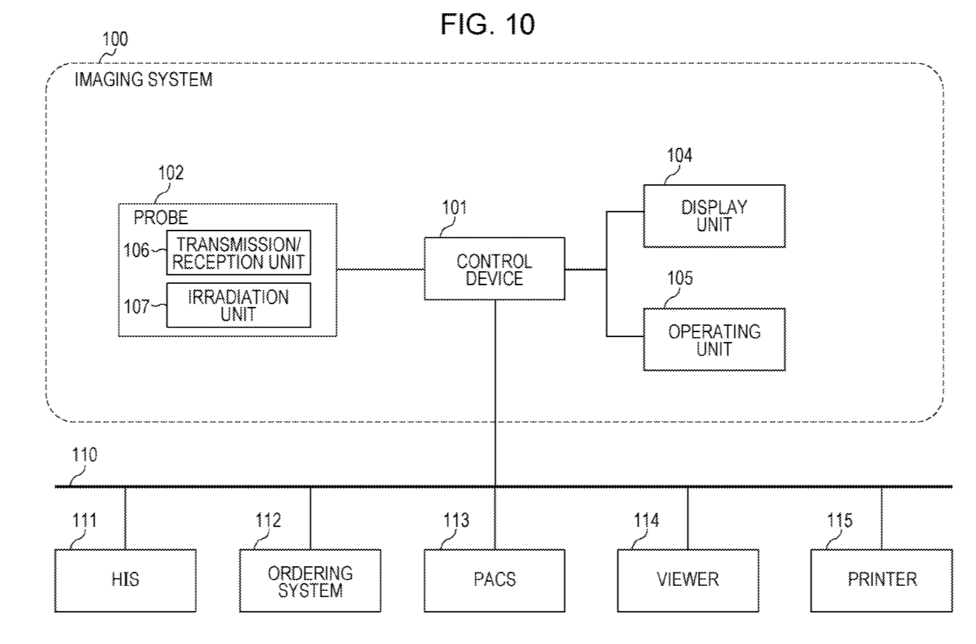

FIG. 10 is a diagram illustrating an example of the configuration of a system including a control device 101 according to the embodiment of the present invention. The imaging system 100 that is capable of generating ultrasound images and photoacoustic images is connected to various types of eternal devices via a network 110. The configurations included in the imaging system 100 and the various types of external devices do not have to be installed within the same facilities, and it is sufficient for these to be communicably connected.

The imaging system 100 includes a control device 101, a probe 102, a display unit 104, and an operating unit 105. The control device 101 is a device that acquires ultrasound wave signals and photoacoustic signals from the probe 102, control acquisition of the photoacoustic signals based on the ultrasound signals for example, and generates photoacoustic images based on this control. The control device 101 also acquires information relating to examinations including imaging of ultrasound images and photoacoustic images from an ordering system 112, and controls the probe 102 and display unit 104 when the examination is performed. The control device 101 outputs generated ultrasound images, photoacoustic images, and superimposed images where photoacoustic images have been superimposed on ultrasound images, to a picture archiving and communication system (PACS) 113. The control device 101 exchanges information with external devices such as the ordering system 112 and PACS 113, in accordance with standards such as Health Level 7 (HL7) and Digital Imaging and Communications in Medicine (DICOM). Details of processing performed by the control device 101 will be described later.

Examples of regions in the body of the subject of examination regarding which ultrasound images are to be taken by the imaging system 100 include the cardiovascular region, breasts, liver, pancreas, abdomen, and so forth. The imaging system 100 may also take ultrasound images of the object where a microbubble-based ultrasound contrast agent has been administered, for example.

Examples of regions in the body of the subject of examination regarding which photoacoustic images are to be taken by the imaging system 100 are regions such as the cardiovascular region, breasts, cervical region, abdomen, the extremities including fingers and toes, and so forth. Particularly, new blood vessels and blood vessel regions including plaque on vascular walls may be the object of taking photoacoustic images, in accordance with characteristics regarding light absorption within the object. Although an example will be described below regarding a case of taking photoacoustic images while taking ultrasound image, the region on the body of the subject where photoacoustic images are being taken by the imaging system 100 does not necessarily have to agree with the region where ultrasound images are being taken. Dyes such as methylene blue, indocyanine green, fine gold particles, and substances where these have been aggregated or chemically modified may be used as a contrast agent in the imaging system 100, and administered to the subject for imaging of photoacoustic images.

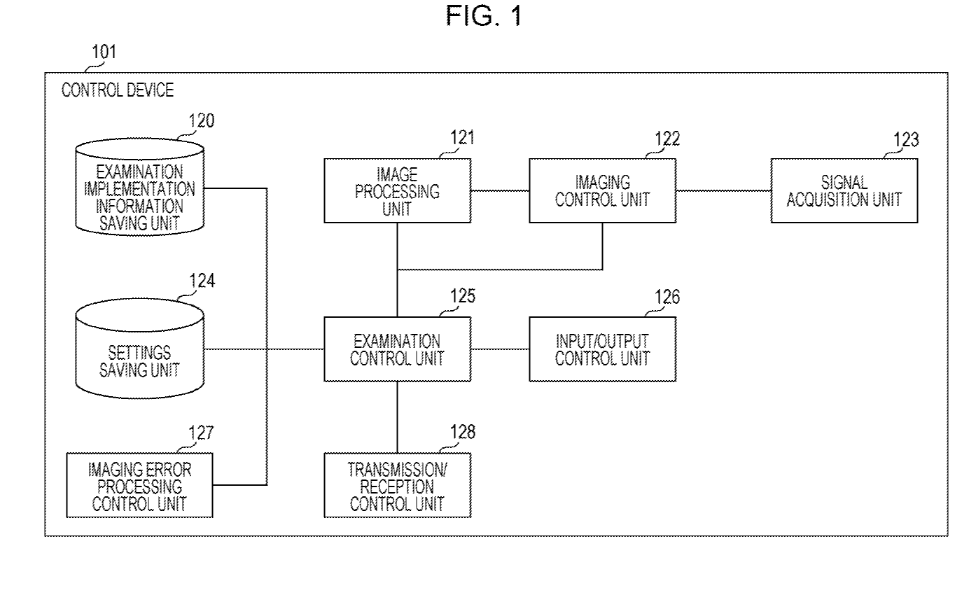

The probe 102 is operated by the user and transmits ultrasound wave signals and photoacoustic signals to the control device 101. The probe 102 includes a transmission/reception unit 106 and an irradiation unit 107. The probe 102 transmits ultrasound waves from the transmission/reception unit 106, and receives the reflected waves at the transmission/reception unit 106. The probe 102 irradiates the subject by light from the irradiation unit 107, and receives the photoacoustic waves at the transmission/reception unit 106. The probe 102 converts the received reflected waves and photoacoustic waves into electrical signals, and transmits to the control device 101 as ultrasound wave signals and photoacoustic signals. The probe 102 is preferably controlled so as to execute transmission of ultrasound waves for acquiring ultrasound wave signals and irradiation by light to acquire photoacoustic signals when information is received that contact has been made with the object. Although one probe is connected to the control device 101 in FIG. 1, multiple probes may be connected.

The transmission/reception unit 106 includes at least one transducer (omitted from illustration), a matching layer (omitted from illustration), a damper (omitted from illustration), and an acoustic lens (omitted from illustration). The transducer (omitted from illustration) is formed of a material that exhibits the piezoelectric effect, such as lead zirconate titanate (PZT) or polyvinylidene difluoride (PVDF). The transducer (omitted from illustration) may be other than a piezoelectric element, and may be a capacitive transducer (capacitive micro-machined ultrasonic transducer (CMUT)) or a transducer using a Fabry-Perot interferometer, for example. Typically, ultrasound wave signals are 2 to 20 MHz and photoacoustic signals are 0.1 to 100 MHz in frequency component, so an arrangement that can detect these frequencies is used for the transducer (omitted from illustration). The signals acquired by the transducer (omitted from illustration) are time-division signals. The amplitude of the received signals represents values based on acoustic pressure received at the transducer at each time. The transmission/reception unit 106 includes a circuit (omitted from illustration) for electronic focus or a control unit. The layout of the transducer (omitted from illustration) is a sector array, linear array, a convex array, an annular array, or a matrix array, for example. The probe 102 acquires ultrasound wave signals and photoacoustic signals, which may be acquired alternately, or acquired simultaneously or may be acquired according to a preset form.

The transmission/reception unit 106 may have an amplifier (omitted from illustration) that amplifies time-sequence analog signals that the transducer (omitted from illustration) has received. The transmission/reception unit 106 may also have an A/D converter that convers time-sequence analog signals that the transducer (omitted from illustration) has received into time-sequence digital signals. The transducer (omitted from illustration) may be divided into a transmitting portion and a receiving portion in accordance with the purpose of imaging the ultrasound image. The transducer (omitted from illustration) may also be divided into a portion for imaging ultrasound images and a portion for imaging photoacoustic images.

The irradiation unit 107 includes a light source (omitted from illustration) for acquiring photoacoustic signals and an optical system (omitted from illustration) that guides pulse light emitted from the light source (omitted from illustration) to the subject. The pulse width of light emitted from the light source (omitted from illustration) is a pulse width of 1 ns or longer to 100 ns or shorter, for example. The wavelength of the light that the light source (omitted from illustration) emits is a wavelength of 400 nm or longer to 1600 nm or shorter, for example. In a case of imaging blood vessels near the surface of the subject with high resolution, a wavelength of 400 nm or longer to 700 nm or shorter, where the absorption at blood vessels is great, is preferable. In a case of imaging deep portions of the body of the subject, a wavelength of 700 nm or longer to 1100 nm or shorter, that is not readily absorbed by water or tissue such as fat, is preferable. Note that the above values are exemplary, and may be changed to other values.

The light source (omitted from illustration) is a laser or light-emitting diode, for example. A light source where wavelength can be changed may be used as the irradiation unit 107, in order to acquire photoacoustic signals using light of multiple wavelengths. Alternatively, the irradiation unit 107 may be of a configuration having multiple light sources that emit light of different wavelengths from each other, where irradiation can be alternately performed by light of different wavelengths from the different light sources. Examples of laser include solid-state laser, gas laser, dye laser, and semiconductor laser. A pulsed laser such as an Nd:YAG laser or an alexandrite laser may be used for the light source (omitted from illustration). Also, a Ti-sapphire laser or optical parametric oscillator (OPO) laser that uses Nd:YAG laser light as excitation light may be used as the light source (omitted from illustration). Further, a microwave source may be used as the light source (omitted from illustration).

Optical elements such as lenses, mirrors, optical fibers, and so forth, are used as the optical system (omitted from illustration). Irradiation is preferably performed with the beam diameter of the pulsed light expanded in a case where the subject is a breast, so a diffraction plate for diffracting the emitted light may be provided to the optical system (omitted from illustration). Alternatively, a configuration may be made where the optical system (omitted from illustration) has lenses or the like and can carry out beam focusing, in order to raise resolution.

The display unit 104 displays images imaged by the imaging system 100 and information relating to the examination, based on control from the control device 101. The display unit 104 provides an interface to accept user instructions, based on control from the control device 101. An example of the display unit 104 is a liquid crystal display.

The operating unit 105 transmits information relating to input of user operations to the control device 101. The operating unit 105 is, for example, a keyboard or trackball, or various types of buttons for performing input of operations relating to examination.

The display unit 104 and operating unit 105 may be integrated as a touch panel display. There is no need for the control device 101, display unit 104, and operating unit 105 to be separate devices, and these configurations may be realized as an integrated console.

A hospital information system (HIS) 111 is a system that supports hospital operations. The HIS 111 includes an electronic health record system, an ordering system, and an medical accounting system. The HIS 111 enables comprehensive management from ordering examinations up to accounting. The ordering system of the HIS 111 transmits order information to ordering systems 112 of respective departments. Implementation of the order is managed at the ordering system 112 described later.

The ordering system 112 is a system that manages examination information, and manages progress of each examination at the imaging device. An ordering system 112 may be configured for each department that performs examination. An example of the ordering system 112 is, in the case of a radiology department, a radiology information system (RIS). The ordering system 112 transmits information for examinations to be performed at the imaging system 100 to the control device 101, in response to a query from the control device 101. The ordering system 112 receives information relating to progress of examinations from the control device 101. Upon having received information from the control device 101 to the effect that an examination has been completed, the ordering system 112 transmits information indicating that this examination has been completed to the HIS 111. The ordering system 112 may be integrated with the HIS 111.

A picture archiving and communication system (PACS) 113 is a database system that stores images obtained at various types of imaging devices inside of and outside of the facilities. The PACS 113 has a storage unit (omitted from illustration) that stores medical images and imaging conditions of the medical images, supplementary information such as parameters of image processing including reconfiguration, patient information, and so forth, and a controller (omitted from illustration) that manages information stored in this storage unit. The PACS 113 stores ultrasound images, photoacoustic images, and superimposed images, which are objects output from the control device 101. Communication between the PACS 113 and the control device 101, and various types of images sored in the PACS 113, preferably conform to standards such as HL7 and DICOM. Various types of images output from the control device 101 are correlated with supplementary information in various types of tags in accordance with the DICOM standard, and stored.

A viewer 114 is an image diagnosis terminal, that reads out images stored in the PACS 113 or the like, and displays for diagnosis. A physician displays images on the viewer 114 and observes the images, and records information obtained as a result of this observation as an image diagnosis report. Image diagnosis reports created using the viewer 114 may be stored in the viewer 114, or may be output to the PACS 113 or a report server (omitted from illustration) and stored.

A printer 115 prints images stored in the PACS 113 or the like. An example of the printer 115 is a film printer, that outputs images stored in the PACS 113 or the like by printing on film.

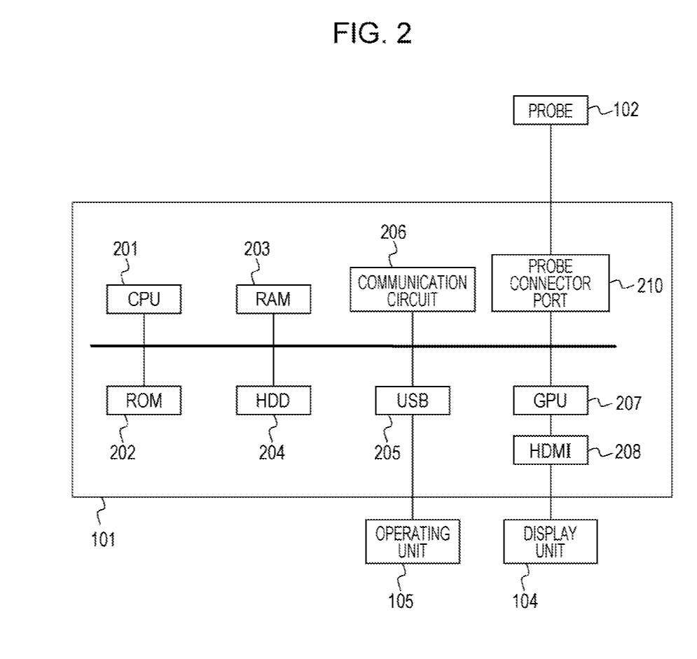

FIG. 2 is a diagram illustrating an example of the configuration of the control device 101. The control device 101 includes a central processing unit (CPU) 201, read-only memory (ROM) 202, random access memory (RAM) 203, a hard disk drive (HDD) 204, a Universal Serial Bus (USB) port 205, a communication circuit 206, a graphics processing unit (GPU) 207, a High-Definition Multimedia Interface (HDMI, a registered trademark) port 208, and a probe connector port 210. These components are communicably connected by a bus. The bus is a data bus that is used for exchanging data among the hardware connected thereto, and for transmitting commands from the CPU 201 to other hardware.

The CPU 201 is a control circuit that centrally controls the control device 101 and components connected thereto. The CPU 201 carries out control by executing programs stored in the ROM 202. The CPU 201 also runs a display driver that is software for controlling the display unit 104, and performs display control of the display unit 104. The CPU 201 further performs input/output control of the operating unit 105.

The ROM 202 stores programs and data recording procedures for control by the CPU 201. The ROM 202 stores a boot program for the control device 101 and various types of initialization data. The ROM 202 also stores various types of programs for realizing the processing by the control device 101.

The RAM 203 provides a workspace storage region for the CPU 201 to perform control by control programs. The RAM 203 has stack and work regions. The RAM 203 stores programs that the control device 101 and components connected thereto executing the processing of, and various types of parameters used in image processing. The RAM 203 stores control programs executed by the CPU 201, and temporarily stores various types of data for the CPU 201 to execute various types of control.

The HDD 204 is an auxiliary storage device for saving various types of data, such as ultrasound images, photoacoustic images, and so forth.

The USB port 205 is a connector to connect to the operating unit 105.

The communication circuit 206 is a circuit for performing communication with components making up the imaging system 100, and various types of external devices connected to the network 110. The communication circuit 206 stores information to be output in the form of transmission packets, and outputs to external devices via the network 110 by communication technology such as TCP/IP, for example. The control device 101 may have multiple communication circuits in accordance with desired communication forms.

The GPU 207 is included in a general-purpose graphics board having video memory. The GPU 207 performs reconstruction processing of photoacoustic images, for example. Using this sort of processing unit enables processing such as reconstruction to be performed at high speeds without requiring dedicated hardware.

The HDMI (registered trademark) port 208 is a connector for connecting to the display unit 104.

The probe connector port 210 is a port for connecting the probe 102 to the control device 101. The ultrasound wave signals and photoacoustic signals output from the probe 102 are acquired by the control device 101 via the probe connector port 210.

The CPU 201 and GPU 207 are examples of a processor. The ROM 202, RAM 203, and HDD 204 are examples of memory. The control device 101 may have multiple processors. In a first embodiment, the functions of components of the control device 101 are realized by the processor of the control device 101 executing programs stored in memory.

Note that the control device 101 may have a CPU or GPU that performs particular processing in a dedicated manner. The control device 101 may also have a field-programmable gate array (FPGA) where particular processing or all processing has been programmed. The control device 101 may have a solid state drive (SSD) as an auxiliary storage device.

FIG. 1 is a diagram illustrating an example of the functional configuration of the control device 101. The control device 101 includes an examination implementation information saving unit 120 (hereinafter referred to as “saving unit 120”), an image processing unit 121, an imaging control unit 122, a signal acquisition unit 123, a settings saving unit 124, an examination control unit 125, an input/output control unit 126, an imaging error processing control unit 127, and a transmission/reception control unit 128.

The saving unit 120 stores information of examinations carried out in the past. Examination information stored in the examination implementation information saving unit 120 is registered, updated, deleted, and searched, based on input of user operations and control from configurations related to the control device 101. The examination implementation information saving unit 120 is made up of a database.

The image processing unit 121 generates ultrasound images and photoacoustic images, and superimposed images where photoacoustic images have been superimposed on ultrasound images. The image processing unit 121 generates ultrasound images to be displayed on the display unit 104 from ultrasound wave signals acquired from the signal acquisition unit 123. The image processing unit 121 generates ultrasound images appropriate for the mode that has been set, based on information of imaging procedures acquired from the examination control unit 125. For example, in a case where the Doppler mode is set as the imaging procedure, the image processing unit 121 generates an image indicating flow rate within the object, based on the difference between the frequency of ultrasound wave signals acquired by the signal acquisition unit 123 and the transmission frequency.

The image processing unit 121 also generates photoacoustic images based on photoacoustic signals acquired by the signal acquisition unit 123. The image processing unit 121 reconstructs distribution of photoacoustic waves when irradiated by light (hereinafter referred to as initial acoustic pressure distribution), based on photoacoustic signals. The image processing unit 121 acquires a distribution of coefficients of light absorption in the object, by dividing the initial acoustic pressure that has been reconstructed by the light fluence distribution in the object of the light that the object has been irradiated by. A concentration distribution of matter in the object is also acquired from distribution of coefficients of light absorption as to multiple wavelengths, using the fact that the degree of absorption of light within the body differs in accordance with the wavelength of light that the body is irradiated by. For example, the image processing unit 121 acquires the concentration distribution of matter in the object regarding oxyhemoglobin and deoxyhemoglobin. The image processing unit 121 further acquires oxygen saturation distribution as a ratio of oxyhemoglobin concentration as to deoxyhemoglobin concentration. Photoacoustic images generated by the image processing unit 121 are images indicating information such as the above-described initial acoustic pressure distribution, light fluence distribution, absorption coefficient distribution concentration distribution of matter, and oxygen saturation distribution, for example.

The image processing unit 121 further composites multiple medical images, and generates a single composite image. For example, an image where a photoacoustic image has been superimposed on an ultrasound image is generated as a composited image. The image processing unit 121 performs image processing for display and diagnosis assistance on the medical images, such as gradient processing. The image processing unit 121 is an example of acquisition means for acquiring ultrasound images and photoacoustic images.

The imaging control unit 122 controls the probe 102 based on information of imaging procedures received from the examination control unit 125. The imaging control unit 122 transmits information relating to the examination order including information of the imaging procedure to the signal acquisition unit 123. The imaging control unit 122 controls the flow relating to acquisition of ultrasound wave signals and acquisition of photoacoustic signals in the examination.

The signal acquisition unit 123 acquires ultrasound wave signals and photoacoustic signals from the probe 102. Specifically, the signal acquisition unit 123 acquires ultrasound wave signals and photoacoustic signals from information acquired from the probe 102, while distinguishing between the two, based on information from the examination control unit 125 and the imaging control unit 122. For example, there are cases where the timing of acquisition of ultrasound wave signals and photoacoustic signals is stipulated in the imaging procedure being used for imaging. In this case, the signal acquisition unit 123 acquires ultrasound wave signals and photoacoustic signals from information acquired from the probe 102, based on the acquisition timing information acquired from the examination control unit 125, while distinguishing between the two.

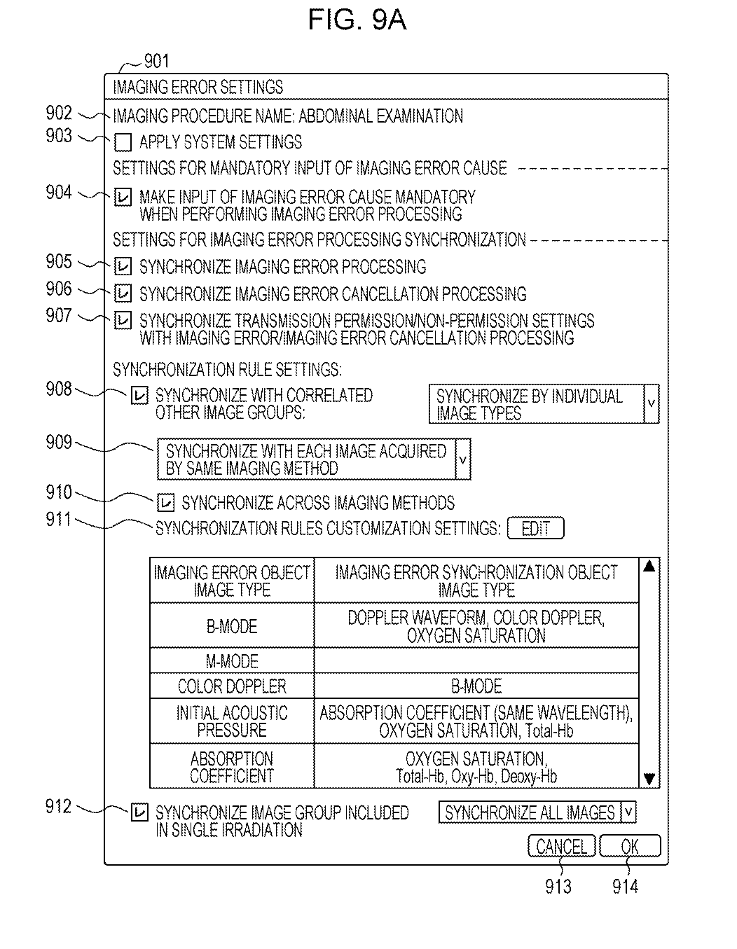

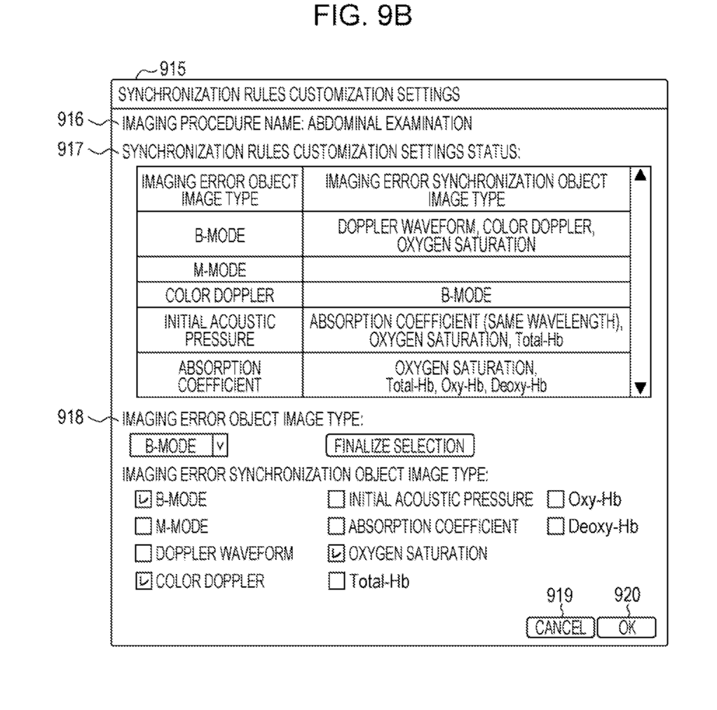

The settings saving unit 124 stores information of settings relating to actions of the control device 101, such as imaging procedure information, settings relating to imaging errors, and so forth. Imaging procedure information is information indicating an imaging procedure performed by the imaging system 100, and information set beforehand regarding the imaging procedure. For example, the settings saving unit 124 accepts and stores setting relating to generating of an object for superimposed display of medical images acquired by the imaging system 100 at an external device. The information of the settings stored in the settings saving unit 124 is registered, updated, deleted, and searched, based on input of user operations and control from configurations related to the control device 101. Details of information of settings stored in the settings saving unit 124 will be described later with reference to FIGS. 9A and 9B. The settings saving unit 124 is made up of a database. The settings saving unit 124 is an example of accepting means.

The examination control unit 125 controls examinations performed by the imaging system 100. The examination control unit 125 acquires information of examination orders from the ordering system 112. Examination orders include information of the patient to be examined, and information relating to the imaging procedure. The examination control unit 125 transmits information relating to the examination order to the imaging control unit 122. The examination control unit 125 also displays information of the examination on the display unit 104 via the transmission/reception control unit 128, to present information relating to the examination to the user. Information of the examination that is displayed on the display unit 104 includes information of the patient to be examined, information of imaging procedures involved in the examination, and images that have already been imaged and generated. The examination control unit 125 further transmits information relating to the progress of the examination to the ordering system 112. For example, when the examination is started by the user, the examination control unit 125 notifies the ordering system 112 of the start, and when imaging by all imaging procedures included in the examination is completed, notifies the ordering system 112 of the completion.

The examination control unit 125 generates information for transmitting various types of information to external devices such as the PACS 113 and viewer 114. For example, the examination control unit 125 generates information to output ultrasound images and photoacoustic images generated at the image processing unit 121, and superimposed images thereof, to the PACS 113. In formation output to external devices include supplemental information supplemented as various types of tags following the DICOM standard. The supplemental information includes patient information, information indicating the imaging device that has imaged this image, image ID for uniquely identifying the image, and examination ID for uniquely identifying the examination where the image was imaged, for example. The supplemental information also includes information correlating ultrasound images and photoacoustic images imaged during the examination. Information correlating ultrasound images and photoacoustic images is information indicating, out of multiple frames making up ultrasound images for example, a frame that is the closest to the timing of having acquired a photoacoustic image. That is to say, the examination control unit 125 generates objects for transmission to external devices based on information stored in the saving unit 120 and settings saving unit 124, and the ultrasound images and photoacoustic images acquired at the image processing unit 121.

The input/output control unit 126 controls the display unit 104 to display information on the display unit 104. From this perspective, the input/output control unit 126 is an example of display control means. The input/output control unit 126 displays information on the display unit 104 in accordance with input from the examination control unit 125 and image processing unit 121, and input of user operations via the operating unit 105.

The imaging error processing control unit 127 performs control relating to imaging error processing based on input of user operations, and control by the examination control unit 125 and information saved in the settings saving unit 124. The imaging error processing control unit 127 controls imaging error processing for specifying image information as being a imaging error, and imaging error cancellation processing for cancelling specification of the imaging error, for example. In imaging error processing of a certain medical image, the imaging error processing control unit 127 selects another medical image that is a different medical image from this medical image, based on supplementary information of this medical image. From this perspective, the imaging error processing control unit 127 is an example of selecting means. The imaging error processing control unit 127 also performs imaging error processing on the other selected medical image. Imaging error processing is processing for attaching information indicating that a medical image is a imaging error to the medical image, for example. From this perspective, the imaging error processing control unit 127 is an example of processing means. Details of processing by the imaging error processing control unit 127 will be described later.

The transmission/reception control unit 128 controls transmission and reception of information among external device such as the ordering system 112, PACS 113, and viewer 114, and the control device 101, via the network 110. The transmission/reception control unit 128 receives information of examination orders from the ordering system 112. The transmission/reception control unit 128 transmits objects generated at the imaging error processing control unit 127 to the PACS 113 and viewer 114.

Note that FIGS. 10, 1, and 2 illustrate an example where the control device 101 is connected to the probe 102 and controls imaging of ultrasound images and photoacoustic images, but the control device according to an embodiment of the present invention is not necessarily restricted to this form. The control device according to the embodiment of the present invention may have a configuration where ultrasound images and photoacoustic images are acquired from a device that controls the imaging.

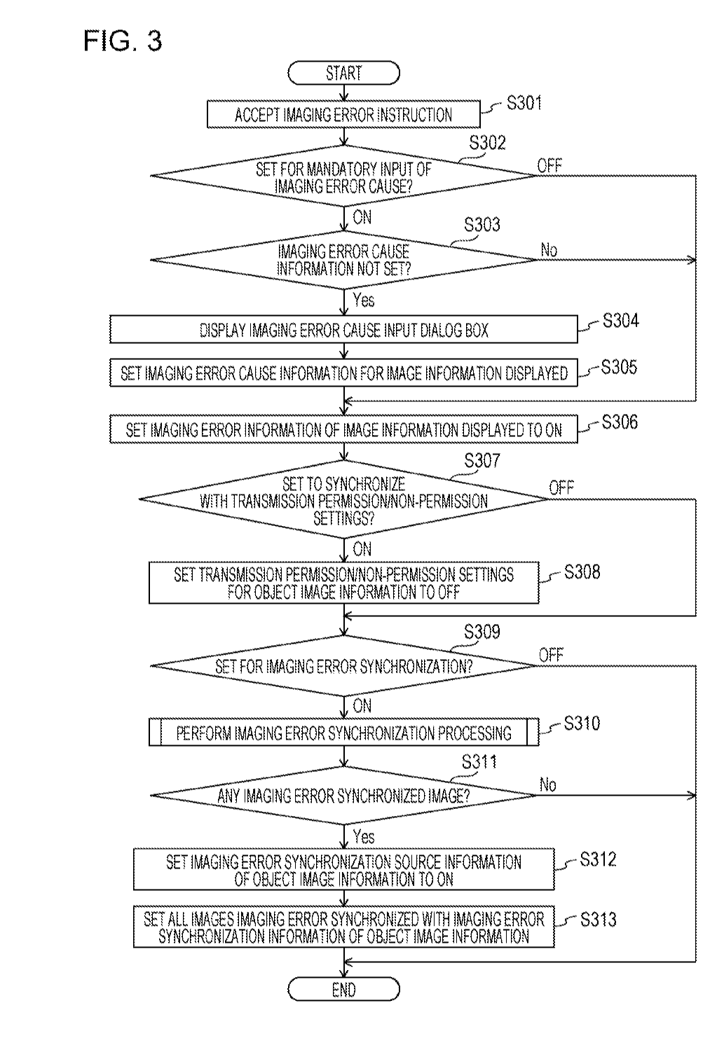

FIG. 3 is a flowchart illustrating an example of processing relating to imaging errors in ultrasound images and photoacoustic images, performed by the control device 101. In the following processing, the entity carrying out the processing in each step is the CPU 201 or the GPU 207, unless particularly stated otherwise.

First, various types of information used in the processing performed by the control device 101 will be described imaging procedure information is information related to imaging procedures carried out by the imaging system 100. Imaging procedure information includes, for example, an imaging procedure ID for uniquely identifying the imaging procedure, the type of modality, imaging part, imaging direction, and information relating to default settings for reconfiguration parameters and imaging conditions. The imaging procedure information also includes settings regarding input of imaging error cause, and information relating to settings for synchronizing imaging error processing among multiple medical images.

Image information is information relating to medical images. Image information includes, for example, information relating to an image ID for uniquely identifying the medical image, the type of modality used for taking this medical image, and image type. The image type indicates the type of medical image that can be taken based on signals acquired in one modality. For example, multiple images such as B-mode images, doppler images, and so forth are acquired based on ultrasound wave signals acquired by an ultrasound imaging device, and information indicating the image type is set thereto. Image information also includes, for example, imaging error information, imaging error cause information, imaging error synchronization source information, imaging error synchronization information, and transmission permission/non-permission information. Image information includes, for example, imaging error information, imaging error cause information, imaging error synchronization source information, and transmission permissible/non-permissible information. Imaging error information is an example of information indicating that a medical image is an imaging error, and is attached to this medical image. Imaging error cause information is information indicating the cause for the medical image being an imaging error. Imaging error synchronization source information is information indicating whether or not imaging error processing of this medical image has been performed in synchronization with imaging error processing performed on another medical image. The imaging error synchronization source information is set to ON for a medical image that has served as a trigger for imaging error processing to be performed on another medical image. The image ID for a medical image regarding which imaging error processing has been performed in synchronization for this medical image is set in the imaging error synchronization information. Further, image information includes superimposed display information, transmission permissible/non-permissible information, image processing parameters, geometric conversion parameters, information relating to objects placed on the medical image, information relating region of interest, and reconstruction parameters. Superimposed display information is information relating to the history of superimposed display performed by the control device 101, and includes, for example, information relating to the form of superimposed display such as layer order and so forth, and image IDs of medical images used for the superimposed display. Objects are, for example, annotation including text input by the user, markings indicating the direction of imaging, information indicating measurement results, and shapes indicating cropping range and masking. The image information may include implemented imaging information indicating the imaging conditions and time of implementation. The implemented imaging information includes image IDs of other medical images correlated with this medical image. For example, medical images generated based on signals acquired within a predetermined period are correlated, based on the signal acquisition time. Medical images handled by the control device 101 include single-frame and multi-frame still images, and moving images.

In step S301, the input/output control unit 126 accepts an imaging error instruction. For example, an imaging error instruction is input through a user interface displayed on the display unit 104. A medical image regarding which an imaging error instruction is accepted is an ultrasound image or a photoacoustic image acquired by the image processing unit 121, for example. In another example, this is a medical image that the input/output control unit 126 has acquired from the PACS 113. From this perspective, the input/output control unit 126 also is an example of acquisition means. The examination control unit 125 acquires image information including the image ID, that is information of uniquely identifying the medial image regarding which there has been the imaging error instruction. Image information here is information including the medial image and metadata correlated with the medical image. The examination control unit 125 transmits the imaging procedure information and image information to the imaging error processing control unit 127, and requests processing for the imaging error.

In step S302, the imaging error processing control unit 127 acquires information set regarding the imaging procedure information acquired in step S301 (settings 904 in FIG. 9A) from the settings saving unit 124. In a case where inputting the cause of the imaging error is mandatory, the flow advances to step S303, and if not mandatory, advances to step S306.

In step S303, the imaging error processing control unit 127 acquires information indicating the cause of the imaging error in the medical image regarding which there has been the imaging error instruction, i.e., an imaging error cause. The imaging error cause is included in image information of the medical image in the present embodiment. In a case where an imaging error cause is included in image information of the medical image, the flow advances to step S306. In a case where no imaging error cause is included in the image information of this medical image, i.e., in a case where imaging error cause is not set in this image information, the flow advances to step S304.

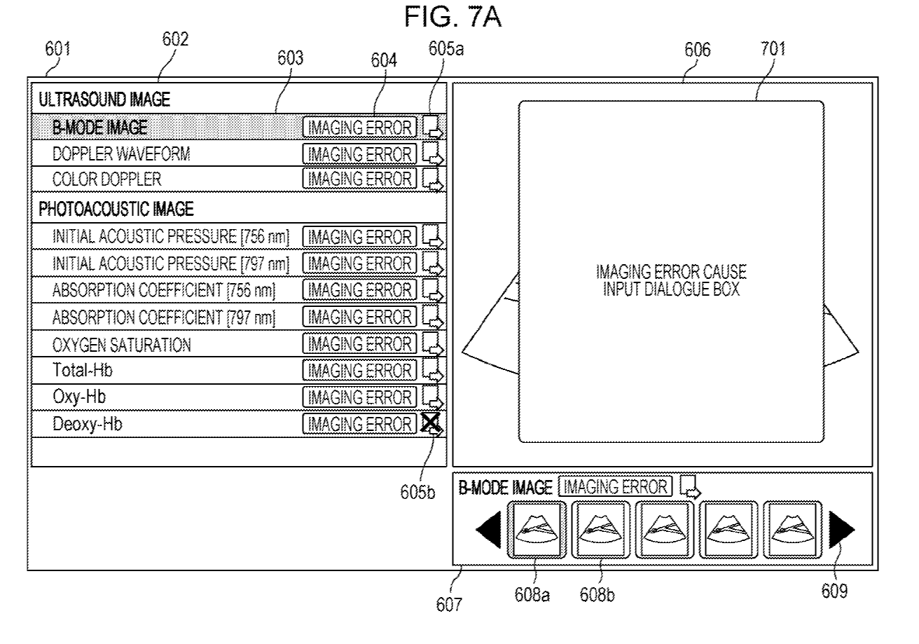

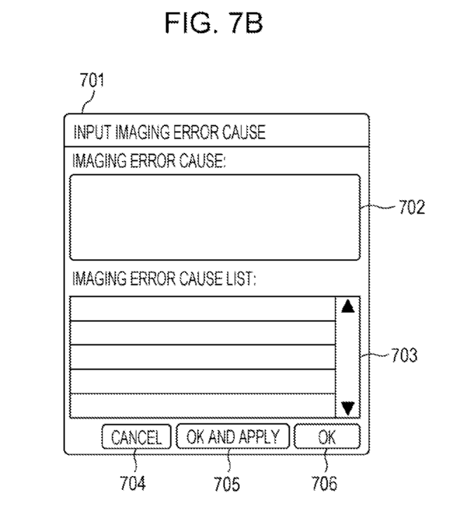

In step S304, the imaging error processing control unit 127 causes an imaging error cause input dialog box 701 (FIG. 7) to be displayed on the display unit 104, via the input/output control unit 126. The imaging error cause input dialog box 701 is a user interface for displaying the cause of the imaging error to the user. The imaging error processing control unit 127 acquires the imaging error cause that the user has input via the imaging error cause input dialog box via the input/output control unit 126.

In step S305, the imaging error processing control unit 127 sets the imaging error cause acquired in step S304 in image information being displayed, i.e., in imaging error cause information in the image information of the medical image regrading which there has been an imaging error instruction.

In step S306, the imaging error processing control unit 127 sets the imaging error instruction to ON in the image information of the medical image regarding which there has been the imaging error instruction.

In step S307, the imaging error processing control unit 127 acquires information of settings related to synchronization of imaging error processing of the medical image and transmission permissible/non-permissible settings, from the settings saving unit 124. In a case where settings relating to transmission permissible/non-permissible are set to synchronize with settings relating to imaging error processing, the flow advances to step S308, and if set to not be synchronized, the flow advances to step S309.

In step S308, the imaging error processing control unit 127 sets transmission permissible/non-permissible settings included in the image information of the medical image that is the object of imaging error instruction to OFF. The transmission permissible/non-permissible settings are information deciding whether transmission of this medical image to an external device is permissible or non-permissible. By the transmission permissible/non-permissible settings bring set to OFF, settings are made such that this imaging error medical image is not transmitted to an external device.

In step S309, the imaging error processing control unit 127 acquires information of settings regarding synchronization of another medical image (imaging error synchronization settings) from the settings saving unit 124. Imaging error synchronization settings are settings deciding, in a case where a medical image taken by a certain imaging procedure is an imaging error, a medical image regarding which to perform imaging error processing synchronously along with imaging error processing of this medical image. In a case where imaging error synchronization settings are ON, the flow advances to step S310, and in a case of OFF, the processing illustrated in FIG. 3 is ended.

In step S310, the imaging error processing control unit 127 performs imaging error processing synchronously with the imaging error processing regarding the medical image regarding which the imaging error instruction has been made in step S301 (imaging error synchronization processing). Details of the processing in step S310 will be described later.

In step S311, the imaging error processing control unit 127 acquires information relating to medical image that has become the object of imaging error synchronization processing in step S310. In a case where a medical image subjected to imaging error synchronization processing exists, the flow advances to step S312, and if there is none, the processing illustrated in FIG. 3 is ended.

In step S312, imaging error synchronization source information of the medical image that is the object of imaging error synchronization processing in step S310 is set to ON.

In step S313, the imaging error processing control unit 127 sets the image IDs of each of the medical images subjected to imaging error processing in the processing up to step S312, in imaging error synchronization information for the medical image regarding which the imaging error instruction was made in step S301. The imaging error synchronization information is included in the image information.

The imaging error processing control unit 127 notifies the examination control unit 125 that processing regarding this imaging error has ended, and the processing illustrated in FIG. 3 is ended.

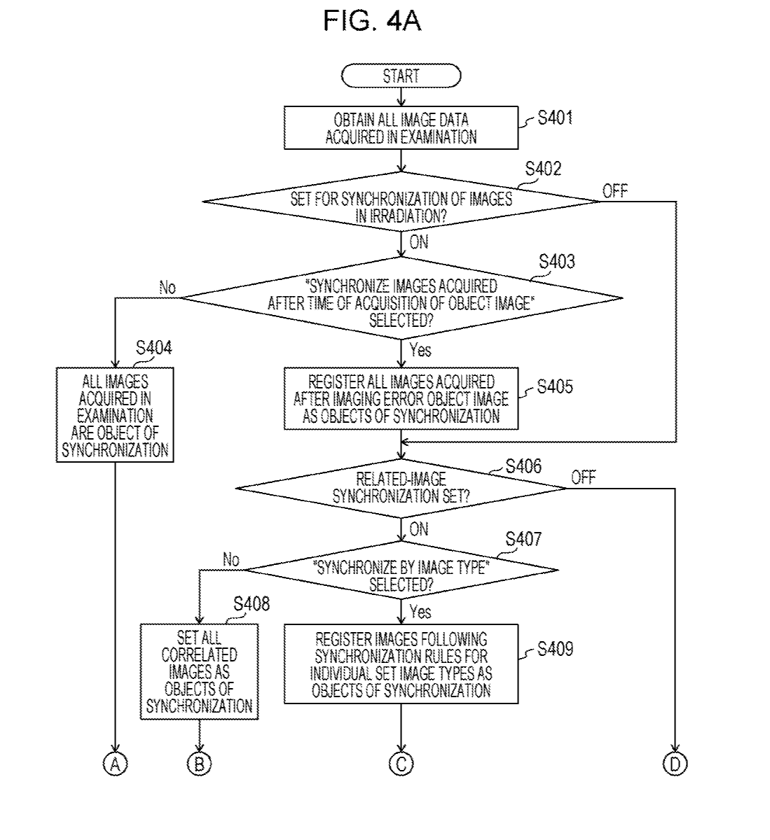

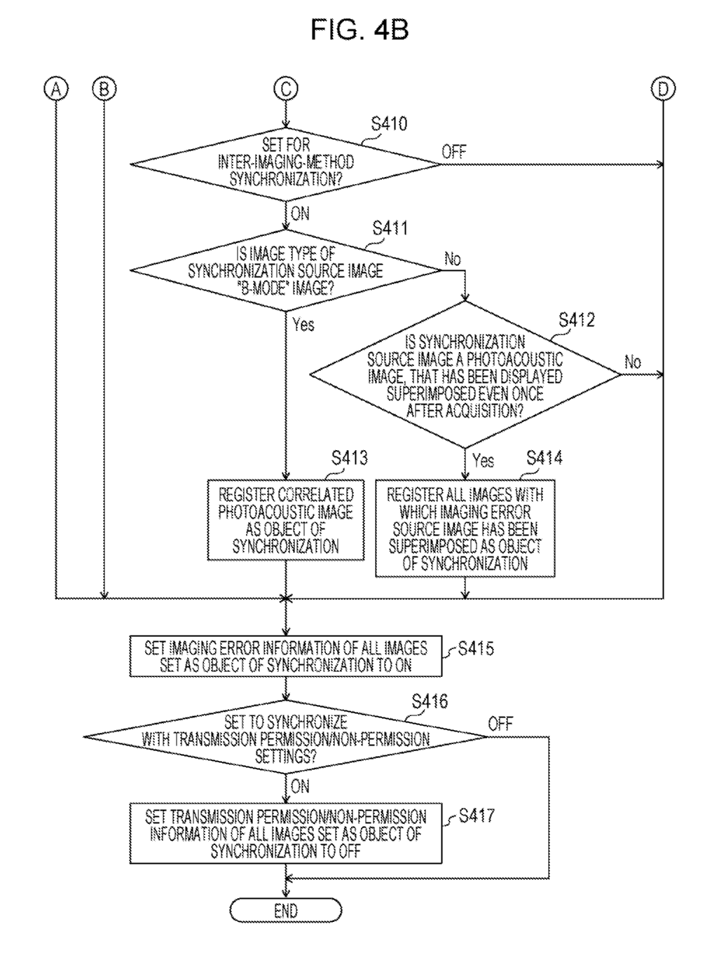

FIGS. 4A and 4B are a flowchart illustrating an example of the imaging error synchronization processing illustrated in step S310 in FIG. 3. In the following processing, the entity carrying out the processing in each step is the CPU 201 or the GPU 207, unless particularly stated otherwise.

In step S401, the imaging error processing control unit 127 acquires, from the saving unit 120, image information of all medical image acquired in the same examination as the medical image regarding which the imaging error instruction was made in step S301 (FIG. 3).

In step S402, the imaging error processing control unit 127 acquires information of settings regarding synchronization of images acquired during one irradiation. First, the imaging error processing control unit 127 searches for image information of medical images acquired at generally the same timing as the medical image regarding which the imaging error instruction was made in step S301 (FIG. 3), out of the image information acquired in step S401. The timing at which medical images are acquired is indicated by image acquisition time included in the implemented imaging information of each image information. The acquisition time is the time at which signals for generating the medical image have been acquired, for example. The acquisition time of an ultrasound images is, in the case of a B-mode image for example, the time at which acquisition of all ultrasound wave signals necessary for generating one B-mode image have been acquired. The time of acquisition of a photoacoustic image is, for example, the time at which acquisition of photoacoustic signals used for generating the photoacoustic image has been completed. The imaging error processing control unit 127 here performs a search regarding image information of medical images of which the image acquisition time is included within a predetermined range as being acquired at generally the same timing, i.e., as having been acquired during one irradiation. That is to say, the imaging error processing control unit 127 searches image information of medical images that have been correlated, according to the example described above with reference to FIG. 3. In a case where image information of a medical image acquired at generally the same timing as a medical image regarding which the imaging error instruction was made in step S301 (FIG. 3) exists, the imaging error processing control unit 127 acquires information of in-irradiation image synchronization settings 912 (FIG. 9) from the settings saving unit 124. The in-irradiation image synchronization settings 912 are settings regarding synchronization of imaging error processing of medical images acquired at generally the same timing. In a case where the in-irradiation image synchronization settings 912 are set to ON, the flow advances to step S403, and if set to OFF, advances to step S406.

In step S403, the processing branches in accordance with the settings of the in-irradiation image synchronization settings 912. In a case where the in-irradiation image synchronization settings 912 are set to “synchronize images acquired after time of acquisition of object image”, the flow advances to step S405, and in a case where “synchronize all” is set, the flow advances to step S404.

In step S404, the imaging error processing control unit 127 takes all image information acquired in step S401 as being the object of imaging error processing synchronously with the imaging error processing of the medical image regarding which the imaging error instruction was made in step S301.

In step S405, the imaging error processing control unit 127 takes image information of a medical image, correlated with the medical image regarding which the imaging error instruction was made in step S301 (FIG. 3) and that is a medical image having a later image acquisition time than the medical image regarding which the imaging error instruction was made, as the object of imaging error processing synchronized with the imaging error processing of the medical image regarding which the imaging error instruction was made. For example, there are cases where the probe 102 is not able to acquire ultrasound wave signals and photoacoustic signals at the same time. As one example, there are cases where the transducer (omitted from illustration) of the probe 102 is not able to distinguish between reflected waves of ultrasound waves that the object has been irradiated by, and photoacoustic waves that the object has been irradiated by, in acquisition. In such a case, images generated from each of ultrasound wave signals and photoacoustic signals acquired within a predetermined time are correlated based on the signal acquisition time, which is to say the image acquisition time. The imaging error processing control unit 127 takes, as the object of imaging error synchronization processing, image information of medical images of which the image acquisition time is later than the medical image regarding which the imaging error instruction was made, out of the correlated medical images, i.e., out of the medical images generated based on signals acquired within the predetermined range of time. Accordingly, medical images acquired later than a medical image that is an imaging error due to body movement of the subject or the like for example, and possibly have been similarly affected by the body movement or the like, can be made to be the object of imaging error synchronization processing.

In step S406, the imaging error processing control unit 127 acquires information relating to correlated image synchronization settings 908 (FIG. 9A) from the settings saving unit 124. In a case where the correlated image synchronization settings 908 is set to ON, the flow advances to step S407, and in a case of being set to OFF, advances to step S415.

In step S407, the processing branches in accordance with the settings of the correlated image synchronization settings 908 (FIG. 9A) acquired in step S406. In a case where the settings of the correlated image synchronization settings 908 are “synchronize by individual image types”, the flow advances to step S409, and in a case where the settings are “synchronize all”, the flow advances to step S408.

In step S408, the imaging error processing control unit 127 takes all medical images correlated with the medical image that is the object of synchronization of imaging error processing, as the object of imaging error processing synchronized with the imaging error processing regarding the medical image regarding which the imaging error instruction was made in step S301.

In step S409, the imaging error processing control unit 127 acquires information relating to image-type-based synchronization rules settings 909 (FIG. 9A), that are settings relating to synchronization rules in accordance with image type, from the settings saving unit 124. The imaging error processing control unit 127 selects the object of imaging error processing synchronized with the imaging error processing regarding the medical image regarding which the imaging error instruction was made in step S301, in accordance with the synchronization rules in accordance with image type.

The settings 909 in the present embodiment can be set to one of “synchronize all images acquired with same imaging method” and “apply customized synchronizing rules”. In a case where “synchronize all images acquired with same imaging method” is selected, medical images acquired by the same imaging method as the medical image regarding which the imaging error instruction was made in step S301 are the object as synchronized imaging error processing. The imaging error processing control unit 127 acquires information relating to the imaging method from image information of the medical images.

For example, assuming that the medical image regarding which the imaging error instruction was made in step S301 is a medical image acquired by a photoacoustic device, in this case, the imaging error processing control unit 127 takes medical images acquired based on photoacoustic signals acquired by the same photoacoustic device, out of the correlated medical images, as the object of imaging error processing synchronized with the imaging error instruction. Medical images indicating information such as, for example, initial acoustic pressure distribution, fluence, absorption coefficient, total hemoglobin amount, oxyhemoglobin amount, deoxyhemoglobin amount, and so forth, can be acquired from photoacoustic signals acquired at generally the same timing by the photoacoustic device. There are cases where multiple photoacoustic images such as described above are obtained based on the same photoacoustic signals, or on photoacoustic signals acquired by irradiation by light of multiple different wavelengths acquired at generally the same timing. In a case where there is an imaging error instruction regarding one of multiple photoacoustic images correlated in this way, there is a possibility that the original photoacoustic signals themselves are to blame, so other photoacoustic images are synchronously made to be the object of imaging error processing. Photoacoustic images generated based on photoacoustic signals obtained by irradiation by different wavelengths may be excluded from being an object of synchronization, or may be displayed on the display unit 104 for the user to judge. In this case, an oxygen saturation image using both photoacoustic signals the same as the photoacoustic image regarding which there has been an imaging error instruction, and photoacoustic signals acquired by irradiation by light of different wavelengths, is taken as the object of imaging error processing synchronized with the imaging error instruction. In a case where there is an imaging error instruction regarding an oxygen saturation image, photoacoustic images acquired based on photoacoustic signals obtained from irradiation by light of all wavelengths used therein.

As another example, a case where there has been an imaging error instruction regarding an ultrasound image in step S301 will be described. For example, the imaging error processing control unit 127 references the image information of an imaging error object, and image information of correlated medical images. In a case where the image type of the ultrasound image that is an imaging error object is a medical image acquired based on intensity (amplitude) of ultrasound wave signals such as with “B-mode”, medical images acquired based on intensity (amplitude) of the same ultrasound wave signals are taken as imaging error objects. For example, medical images based on M-mode where measurement values of a particular position are recoded under observation in B-mode, or A-mode where distance (time) and intensity (amplitude) of ultrasound wave signals are recorded, are synchronously taken by the imaging error processing control unit 127 as being imaging error objects. On the other hand, medical images using frequency shift due to movement of the source of observer, such as in the doppler mode, are medical images acquired by a different imaging method from medical images in B-mode, and accordingly are excluded from being objects of synchronization with imaging error instruction. Medical images of image types such as “continuous doppler”, “pulsed doppler”, “color doppler”, and so forth, that are acquired based on the same frequency shift information, are taken by the imaging error processing control unit 127 as being imaging error objects synchronously with an imaging error instruction regarding one thereof.

In a case where the settings 909 are set to “apply customized synchronizing rules”, the imaging error processing control unit 127 obtains information relating to customized synchronizing rules from the settings saving unit 124. The customized synchronizing rules are selected at the settings 911 (FIG. 9A), for example. According to the example illustrated in FIG. 9A, in a case where the image type is “B-mode” regarding a medical image regarding which there has been an imaging error instruction, “doppler waveform”, “color doppler”, and “oxygen saturation” are set as image types for which other medical images are synchronously taken as the object of imaging error processing, out of the correlated medical images. Details of customized synchronizing rules are set by a screen exemplified in FIG. 9B.

In step S410, in a cases where settings 908 are set to “synchronize by individual image types”, the imaging error processing control unit 127 further acquires information relating to settings 910 (FIG. 9A) for synchronization in a case where the imaging methods differ, from the settings saving unit 124. In a case where the settings 910 are set to ON, the flow advances to step S411, and in a case of being set to OFF, the flow advances to step S415.

The flow branches at step S411 in accordance with the type of image of the image information for the medical image regarding which the imaging error instruction was made in step S301. The imaging error processing control unit 127 acquires information relating to the image type. In a case where this image type is “B-mode”, the flow advances to step S413, and if other than “B-mode”, advances to step S412.

In step S413, the imaging error processing control unit 127 sets the photoacoustic image correlated with the “B-mode” ultrasound image regarding which the imaging error instruction was made, synchronously as an object of imaging error processing.

The imaging error processing control unit 127 in step S412 acquires information related to history of superimposed display at the control device 101 of the medical image regarding which the imaging error instruction was made in step S301. Note that in the present embodiment, the imaging error processing control unit 127 acquires superimposed display information included in the image information of the medical image regarding which the imaging error instruction was made. In a case where this medical image regarding which the imaging error instruction was made is a photoacoustic image, and has history of having been displayed superimposed, the flow advances to step S414, and otherwise, advances to step S415.

In step S414, the imaging error processing control unit 127 takes the medical image registered in the superimposed display information as the object of imaging error processing synchronously with the medical image regarding which the imaging error instruction was made in step S301.

For example, a photoacoustic image and ultrasound image may be displayed superimposed to be used for interpretation. Accordingly, in a case where one of the medical images is a imaging error, there are cases where performing interpretation using the other medical image alone is difficult. In a case where one of the medical images of the superimposed display is found to be a imaging error, the other medical image may also be handled as being a imaging error, in a synchronized manner, due to the processing of steps S411 through S414. In a case where the user desires to perform interpretation by the ultrasound image alone, the processing of steps S412 through S414 may be skipped.

In step S415, the imaging error processing control unit 127 sets the imaging error information, for all medical images that have been found to an imaging error object in the processing up to step S414, to ON. The imaging error processing control unit 127 obtains information relating to the settings for the imaging error cause for each medical image that is the object of imaging error processing. In a case where input of the imaging error cause is set to mandatory, an imaging error cause input dialog box (FIG. 7) is displayed on the display unit 104 via the input/output control unit 126. For image information of medical images set to imaging errors synchronously with the medical image regarding which the imaging error instruction was made in step S301, the imaging error processing control unit 127 may automatically record information that these medical images have been set to imaging error synchronously with another medical image.

The imaging error processing control unit 127 acquires information relating to settings of synchronization between transmission permission/non-permission settings and imaging error processing from the settings saving unit 124 in step S416. The settings of synchronization between transmission permission/non-permission settings and imaging error processing are set via the settings 907 illustrated in FIG. 9A, for example. In a case where there is to be synchronization between transmission permission/non-permission settings and imaging error processing, the flow advances to step S417, and in a case of not synchronizing, the processing illustrated in FIGS. 4A and 4B is ended.

In step S417, the imaging error processing control unit 127 sets the transmission permission/non-permission information of medical images that have become the object of imaging error processing in the processing up to step S414 to OFF. Accordingly, the control device 101 ends the processing illustrated in FIGS. 4A and 4B.

According to the processing illustrated above, the control device 101 according to the embodiment of the present invention can automatically perform imaging error processing on other medical images regarding which imaging error processing should be performed in conjunction, synchronously with imaging error instructions regarding a certain medical image, and thus improve efficiency of the work for the user to perform.

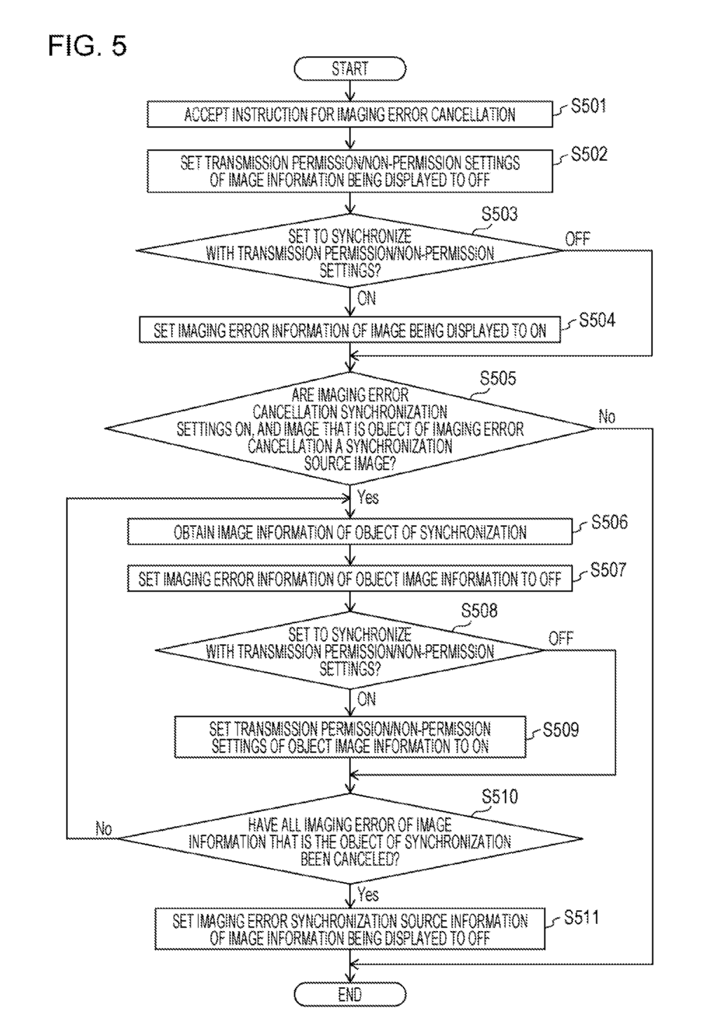

FIG. 5 is a flowchart illustrating an example of processing for cancelling imaging error status, performed by the control device 101. In the following processing, the entity carrying out the processing in each step is the CPU 201 or the GPU 207, unless particularly stated otherwise.

In step S501, the input/output control unit 126 accepts instructions from the user to cancel imaging error status. This imaging error cancellation is performed by input having been performed via the operating unit 105 to the interface displayed on the display unit 104, for example. The examination control unit 125 obtains the image ID of the medical image regarding which imaging error cancellation has been instructed. The examination control unit 125 also obtains the imaging procedure information and image information of the medical image regarding which imaging error cancellation has been instructed, from the saving unit and settings saving unit 124, and transmits to the imaging error processing control unit 127. The examination control unit 125 requests the imaging error processing control unit 127 for cancellation of the imaging error status of the medical image identified by the imaging procedures information and the image information including the image ID.

In step S502, the imaging error processing control unit 127 sets the imaging error instruction included in the image information of the medical image regarding which imaging error cancellation has been instructed in step S501 to OFF.

In step S503, the imaging error processing control unit 127 obtains information relating to settings 907 (FIG. 9A) for synchronization of transmission permission/non-permission settings and imaging error processing from the settings saving unit 124. In a case where the settings 907 are set to ON, the flow advances to step S504, and in a case of being set to OFF, the flow advances to step S505.

In step S504, the imaging error processing control unit 127 sets the transmission permission/non-permission information in the image information of the medical image regarding which imaging error cancellation has been instructed in step S501 to ON.

In step S505, the imaging error processing control unit 127 obtains information relating to settings 906 (FIG. 9A) regarding synchronization of imaging error cancellation processing and cancellation of the imaging error status of other medical images, from the settings saving unit 124. The imaging error processing control unit 127 further obtains imaging error synchronization source information included in the image information of the medical image regarding which imaging error cancellation has been instructed in step S501 described in FIGS. 3 through 4B. A medical image regarding which the imaging error synchronization source information is ON means that this is a medical image regarding which an imaging error instruction was made in step S301, and that this is a medical image that has triggered synchronized imaging error processing of another medical image. In a case where the settings 906 are ON and the imaging error synchronization source information of the medical image regarding which imaging error cancellation has been instructed in step S501 is ON, the flow advances to step S506. If the settings are OFF or the imaging error synchronization source information is OFF, the control device 101 ends the processing illustrated in FIG. 5.

In step S506, the imaging error processing control unit 127 obtains the imaging error synchronization information included in the image information of the medical image regarding which imaging error cancellation has been instructed in step S501. The imaging error synchronization information identifies medical images that have been synchronously subjected to imaging error processing.

In step S507, the imaging error processing control unit 127 sets the imaging error information of the medical images identified in step S506 to OFF.

In step S508, the imaging error processing control unit 127 obtains information relating to the settings 907 (FIG. 9A) for synchronization of imaging error cancellation processing and transmission permission/non-permission settings, from the settings saving unit 124. In a case where the settings 907 are ON, the flow advances to step S509, and in a case of being OFF, advances to step S510.

In step S509, the imaging error processing control unit 127 sets the transmission permission/non-permission information of the medical image identified in step S506 to OFF.

In step S510, the imaging error processing control unit 127 obtains imaging error information of the medical images that have become the object of imaging error cancellation in the processing up to step S509. In a case where the imaging error information of all medical images that are the object of imaging error cancellation is OFF, and cancellation of the imaging error status has been completed, the flow advances to step S511. In a case where imaging error cancellation has not been completed, the flow advances to step S506 and repeats the above-described processing.

In step S511, the imaging error processing control unit 127 sets the synchronization source information of the medical image regarding which imaging error cancellation was instructed in step S501, and deletes all image IDs registered in the imaging error synchronization information.

Thus, the processing illustrated in FIG. 5 is ended. The imaging error processing control unit 127 notifies the examination control unit 125 that the imaging error cancellation processing has been completed. Accordingly, the control device 101 that can perform imaging error processing as to a certain medical image synchronously with regard to another medical image as well, can perform imaging error cancellation processing as to a certain medical image synchronously with regard to another medical image as well, in the same way, and thus can save trouble for the user.

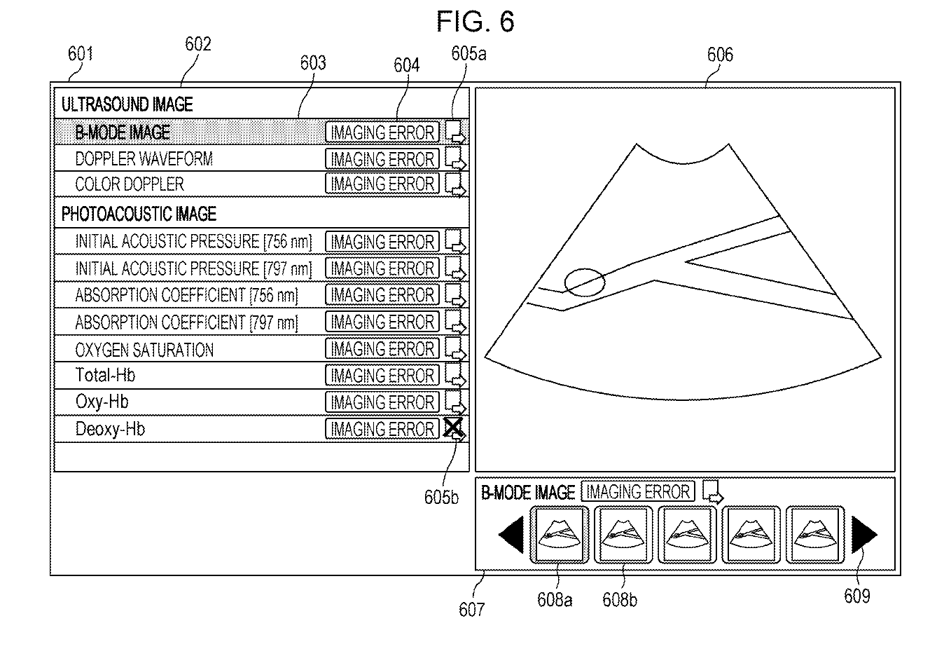

FIG. 6 is an example of an interface for performing imaging error instruction and cancellation as to a medical image acquired by the imaging system 100. A superimposed image where a photoacoustic image has been superimposed on an ultrasound image acquired by the imaging system 100 is illustrated in the example in FIG. 6.

An examination screen 601 includes a related image list 602, related image items 603, imaging error instruction portions 604, transmission instruction portions 605, an image display portion 606, a thumbnail image display portion 607, thumbnail images 608, and a page switching instruction portion 609.

The related image list 602 is an image list of all images related with a thumbnail image 608 in a selected state in the thumbnail image display portion 607. In a case where all information in the related image list 602 cannot be displayed on the display unit 104, the display of the related image list 602 is switched by scrolling. A related image item 603 is displayed on each row of the related image list 602. Although the related image list 602 is displayed sorted by imaging type in the example illustrated in FIG. 6, the related image items 603 corresponding to images imaged in correlation with the examination may be displayed included in a single list.

The related image items 603 is a list item corresponding to a medical image in a one on one manner. Text strings indicating the name of the medical images, imaging error instruction portions 604, and transmission instruction portions 605 are displayed in the related image items 603. Thumbnail images of medical images may be displayed on the related image items 603 instead of the text strings indicating the name of the medical images, or both the text strings and the thumbnail images may be displayed. The user can select a related image item 603 by performing input of operations regarding the related image items 603. A medical image corresponding to the selected related image item 603, or a medical image related to this medical image, is displayed in the image display portion 606.

The imaging error instruction portion 604 is a button for performing imaging error processing on the medical image corresponding to the related image item 603. When imaging error processing is executed, imaging error information 306 corresponding to this medical image is switched to ON. When the imaging error status is cancelled, the imaging error information included in the image information of this medical image is switched to OFF.

The transmission instruction portion 605 is a button for individually instructing whether or not to transmit the medical image corresponding to the related image item 603 to an external device. When the transmission instruction portion 605 is set to on, the transmission permission/non-permission information corresponding to this medical image is switched to ON. The transmission instruction portion 605 is displayed in different forms according to the ON and OFF settings. In the example in FIG. 6, the transmission instruction portion 605 is displayed as indicated by the transmission instruction portion 605a in a case of ON, and is displayed as indicated by the transmission instruction portion 605b if OFF.

Thus, imaging error processing and transmission to external devices can be set for each related image item 603 in the control device 101, i.e., for each medical image. Allowing the imaging error instruction portion 604 and transmission instruction portion 605 to be set separately allows the control device 101 to distinguish whether the reason that transmission to an external device is not performed is because of a imaging error or not. Note that in the example in FIG. 6, the imaging error instruction portion 604 and transmission instruction portion 605 are displayed and can be set for all related image items 603 in the related image list 602. However, this example is not restrictive, and an arrangement may be made where, for example, the imaging error instruction portion 604 and transmission instruction portion 605 are displayed and are settable only for the related image item 603 corresponding to the medical image being displayed on the image display portion 606.

The image display portion 606 is a region for displaying medical images acquired by the imaging system 100. The images displayed in the image display portion 606 may be of any image type, such as still images, moving images, waveform data, and so forth. While imaging a moving image, a preview of the image is displayed in real-time in the image display portion 606. A preview of a medical image corresponding to a related image item 603 selected from the related image list 602 is displayed in the image display portion 606.

The thumbnail image display portion 607 is a region where thumbnail images corresponding to a medical image selected as a reference out of the multiple medical images correlated in the examination. The thumbnail image display portion 607 includes an imaging error instruction portion 604, transmission instruction portion 605, thumbnail images 608, and page switching instruction portion 609. In the example illustrated in FIG. 6, thumbnail images of a series of B-mode images that are an example of ultrasound images, are displayed in the time-sequence of acquisition.

Displayed in the thumbnail image display portion 607 are thumbnail images 608 corresponding to respective medical images, for every group of medical images acquired in one imaging procedure, for example. In a case where a moving image is acquired, a thumbnail image 608 corresponding to a representative frame image is displayed out of the multiple frames included in the moving image. Thumbnail images 608 corresponding to sequentially acquired medical images are added to the thumbnail image display portion 607 on the examination screen 601 each time a medical image is acquired, until the examination ends.

The medical image to be displayed in preview in the image display portion 606 is selected in accordance with input of operations on the thumbnail images 608 displayed in the thumbnail image display portion 607. A thumbnail image 608 that has been selected is in a preview selection state (608a), and a thumbnail image 608 that has not been selected is in a non-preview selection state (608b). In accordance with thumbnail images 608 to be displayed in preview in the image display portion 606 having been switched, the display of the imaging error instruction portion 604 included in the thumbnail image display portion 607, the transmission instruction portion 605, and the related image items 603 displayed in the related image list 602, are updated. The imaging error instruction portion 604 and transmission instruction portion 605 included in the thumbnail image display portion 607 indicate the imaging error settings and transmission permissible/non-permissible settings regarding the selected thumbnail image 608a. In a case where not all thumbnail images 608 can be displayed in the thumbnail image display portion 607, the page switching instruction portion 609 is enabled. Pressing the page switching instruction portion 609 displayed to the right and left sides of the thumbnail image 608 group switches the list of thumbnail images 608 displayed in the thumbnail image display portion 607.

FIGS. 7A and 7B illustrate an example of the imaging error cause input dialog box 701 prompting the user to input a cause of a imaging error. Upon an imaging error instruction being given using the imaging error instruction portion 604 illustrated in FIG. 6, a pop-up display of the imaging error cause input dialog box 701 is made on the examination screen 601 (FIG. 7A). In a case where settings 904 relating to input of the imaging error cause are set to OFF, the dialog box 701 is not displayed. The size and position of display of the imaging error cause input dialog box 701 can be optionally changed. The dialog box 701 has an input portion 702, a selection portion 703, a cancel portion 704, an application instruction portion 705, and an OK portion 706 (FIG. 7B).

The input portion 702 is a region where the user inputs and edits the imaging error cause. Note that in a case where the imaging error cause is set in the image information of the imaging error object, the imaging error cause set in the input portion 702 is displayed.

The selection portion 703 is a region for the user to select an imaging error cause from imaging error causes that have been registered beforehand. The user can set beforehand text strings representing imaging error causes, and the display order in the selection portion 703. The contents that have been set are saved in the settings saving unit 124. Selecting an imaging error cause from the list in the selection portion 703 inputs the selected imaging error cause to the input portion 702.

The cancel portion 704 is a button to instruct discarding of the contents of the imaging error cause that have been input. Operating input of the cancel portion 704 closes the dialog box 701.

The application instruction portion 705 is a button to finalize the imaging error cause input to the input portion 702, and further to instruct applying this finalized imaging error cause as the imaging error cause for other medical images as well. Accordingly, the same imaging error cause as with the imaging error instruction as to a certain medical image is input for another medical image subjected to imaging error processing synchronously. When the imaging error cause is finalized, the dialog 701 is closed.

The OK portion 706 is a button instructing finalizing of the imaging error cause input to the input portion 702. Upon the imaging error cause having been finalized, the dialog 701 is closed.

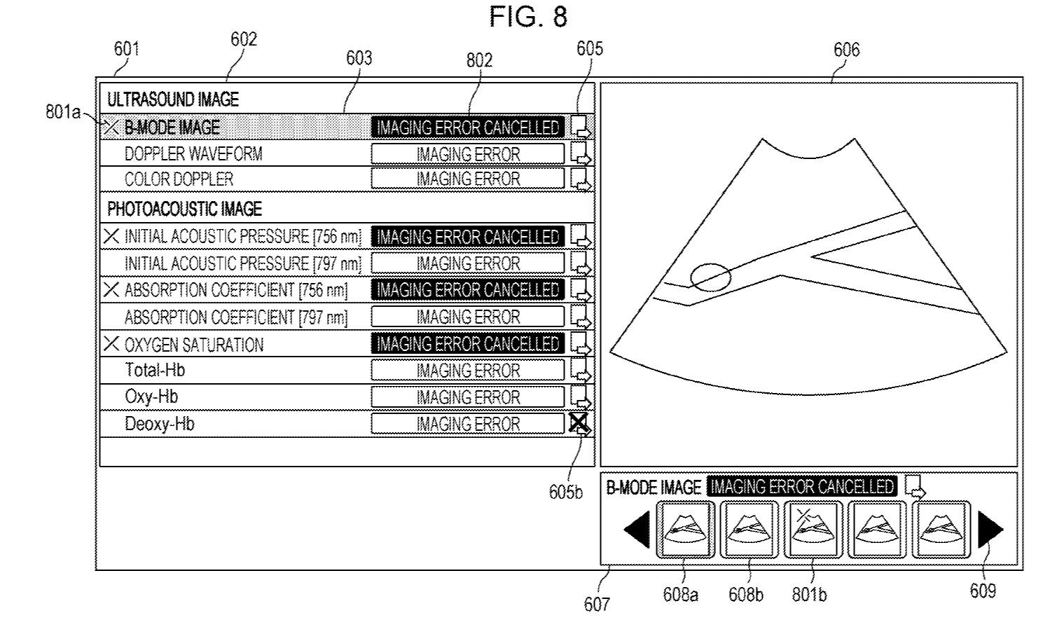

FIG. 8 is an example of the examination screen 601 after imaging error processing has been performed regarding a certain medical image. An imaging error mark 801a is displayed by related image items 603 regarding which imaging error processing has been performed. The imaging error instruction portion 604 switches to an imaging error cancellation portion 802. The imaging error cancellation portion 802 is a button for instructing cancellation of the imaging error status of a medical image regarding which an imaging error instruction has been given. When processing for cancelling the imaging error status is performed, the processing in FIG. 5 is carried out. The imaging error mark 801a is no longer displayed by the related image item 603 of a medical image regarding which the imaging error status has been cancelled. The imaging error cancellation portion 802 switches to the imaging error instruction portion 604. In a case of having given an imaging error instruction to an thumbnail image within the thumbnail image display portion 607, an imaging error mark 801 (thumbnail 801b) is displayed on the object thumbnail image.

FIG. 9 is an example of a settings screen 901 for making various types of settings regarding imaging errors. The information set in the settings screen 901 is saved in the settings saving unit 124. A region 902 displays the name of the imaging procedure regarding which settings are to be made. Although an example of a screen where various settings are made regarding a certain imaging procedure is shown here, an arrangement may be made where various settings can be made in batch fashion for the processing of the control device 101.

Settings 903 are setting for selecting whether or not to perform batch settings at the control device 101. When the settings 903 are set to ON, system settings are applied to imaging error processing on medical images acquired by the imaging procedure displayed in the region 902. System settings are settings decided beforehand separately from the settings shown in FIGS. 9A and 9B, and are applied to imaging error processing for all imaging procedures where the settings 903 are set to ON. When the settings 903 are set to OFF, individual settings such as described below are applied to the imaging procedure shown in the region 902.

The settings 904 are settings relating to input of the imaging error cause. When the settings 904 are set to ON, the dialog box 701 (FIGS. 7A and 7B) that is an interface for the user to input the imaging error cause is displayed on the display unit 104. Imaging error processing is performed after the imaging error cause is input to the dialog box 701 and finalized.

Settings 905 are settings for selecting whether or not imaging error processing for a certain medical image is to be synchronously applied to another medical image. When the settings 905 are set to ON, imaging error processing is performed synchronously with other medical images, based on the various types of settings described below.

The settings 906 are settings for selecting whether or not imaging error cancellation processing for a certain medical image is to be synchronously applied to another medical image. When the settings 906 are set to ON, in a case where the imaging error processing for the medical image which is the object of the imaging error cancellation has been performed synchronously with other medical images, the imaging error cancellation processing is performed synchronously with the other medical images in the same way.

The settings 907 are settings for selecting, when imaging error processing or imaging error cancellation processing has been instructed regarding a certain medical image, whether or not to synchronously switch transmission permissible/non-permissible settings for this medical image. When the settings 907 are set to ON, the transmission permissible/non-permissible settings are automatically switched so that a medical image that has been given an imaging error status is not transmitted to an external device, and a medical image regarding which the imaging error status has been cancelled is transmitted to an external device.

The settings 908 are settings for synchronizing imaging error processing and imaging error cancellation processing regarding a certain image with other medical images correlated with this medical image. Further, rules for synchronizing with other medical images correlated with this medical image can be set. In the example illustrated in FIG. 9A, selection can be made from options including at least “synchronize all” and “synchronize by individual image types” regarding rules for synchronizing. The contents of the settings 908 have been described above, so detailed description will be omitted hereby referencing the previous description.

The settings 909 are settings for selecting synchronizing rules for individual image types. In the example illustrated in FIG. 9A, selection can be made from options including at least “synchronize all images acquired with same imaging method”, “synchronize by image types acquired with same imaging method”, and “apply customized synchronizing rules”. In a case where “synchronize all images acquired with same imaging method” is selected, all image groups acquired by the same imaging method as the object image, out of other images correlated with the image that is the object of imaging error processing or imaging error cancellation processing, are synchronized. Detailed description of the contents of the settings 909 will be omitted by referencing the previous description.

The settings 910 are settings for selecting whether or not to synchronize medical images correlated with imaging error processing or imaging error cancellation processing but have been acquired by a different imaging method. Detailed description of the contents of the settings 910 will be omitted by referencing the previous description.

The settings 911 are settings for the user to customize synchronizing rules. In the example illustrated in FIG. 9A, settings are shown separately for each image type of ultrasound images and photoacoustic images, to handle a device that is capable of acquiring ultrasound wave signals and photoacoustic signals. When the user presses an edit button in the settings 911, a dialog box 915 for custom settings is displayed on the display unit 104.

FIG. 9B is an example of the dialog box 915 for custom settings. The name of the imaging procedure regarding which settings are to be made is displayed in a region 916. A settings display portion 917 displays a list of the contents of settings that have been input, sorted by image type.

Settings 918 are a region where settings regarding synchronization of imaging error processing and imaging error cancellation processing are made for each image type. An image type to be the object of editing is selected. Image types regarding which imaging error processing and imaging error cancellation processing is to be synchronized with medical images of this selected image type are then selected. A cancel portion 919 discards the contents of editing, and an OK portion 920 finalizes the contents of editing. Cancelling or finalizing the contents of editing closes the dialog box 915, and the contents thereof are reflected in the settings 911.

Settings 912 are settings for selecting whether or not to synchronize imaging error processing and imaging error cancellation processing among medical images included in one irradiation, i.e., among multiple medical images that have been correlated. When the settings 912 are set to ON, synchronized processing is performed among the multiple medical images that have been correlated, following synchronizing rules described below. The synchronizing rules can be selected from options including at least “synchronize all” and “synchronize images acquired after time of acquisition of object image”. In a case where “synchronize all” is selected, synchronized processing is performed on all of the multiple medical images that have been correlated, including a medical image that is the object of imaging error processing or imaging error cancellation processing. In a case where “synchronize images acquired after time of acquisition of object image” has been selected, synchronized processing is performed on, out of the multiple medical images that have been correlated including a medical image that is the object of imaging error processing or imaging error cancellation processing, medical images acquired after the medical image that is the object.

A cancel portion 913 discards the contents of editing, and an OK portion 914 finalizes the contents of editing.

### Modification

Although an example of synchronizing imaging error instructions and permission/non-permission regarding transmission of the medical image regarding which the imaging error instruction has been made to an external device has been described in the above embodiment, the present invention is not restricted to this, and an imaging error status and permission/non-permission of transmission do not necessarily have to be synchronized.

An example has been described in the above embodiment where imaging error processing is automatically performed in a case where, in accordance with an imaging error instruction regarding a certain medical image, imaging error processing is synchronously performed regarding another medical image. The present invention is not restricted to this, and an arrangement may be made where, in a case that the control device 101 has determined synchronized imaging error processing should be performed, for example, the user is presented with an interface to select whether or not imaging error processing should be performed regarding medical images that are the object of this synchronized imaging error processing. Work where the user observes all medical images acquired in an examination and determines whether or not a imaging error can be troublesome work for the user. Presenting medical images that may be imaging errors in this way improves the workflow for the user.

Description has been made in the above embodiment regarding an example where, in accordance with an instruction to cancel the imaging error status of a certain medical image, imaging error cancellation processing of this image is synchronously performed regarding another medical image that has been determined to be a imaging error. The present invention is not restricted to this, and an arrangement may be made where the user is presented with an interface to select whether or not imaging error cancellation processing should be performed regarding medical images that are the object of this synchronized imaging error cancellation processing.

Although an example of performing the processing illustrated in FIGS. 4A and 4B has been described in the above embodiment, the present invention is not restricted to this. For example, synchronized imaging error processing may be performed for all medical images acquired at generally the same timing and identified in step S402. An arrangement may also be made where, instead of performing the processing of step S403, determination is made regarding whether or not to synchronize imaging error processing based on conditions set regarding image types and imaging methods out of all images acquired in the examination. That is to say, an arrangement may be made where the processing described in step S407 or step S410 is performed after step S404. Further, in the processing illustrated in FIGS. 4A and 4B, an arrangement may be made where the processing of none of steps S403, S407, and S410 is performed, or where only one, or where a combination of any two, is performed.

Although an example has been described in the above embodiment where imaging error processing is synchronized based on information attached to medical images, such as image type and acquisition time, the present invention is not restricted to this. For example, medical images to be synchronized may be selected based on the input of the imaging error cause in imaging error processing of a certain medical image. In a case where the subject having moved while imaging has been input as the imaging error cause, medical images acquired after the medical image that has been determined to be a imaging error may be taken as imaging errors in a synchronized manner. In another example, in a case where insufficient signal value intensity has been input as the imaging error cause, all medical images acquired in the examination may be taken as imaging errors. In yet another example, in a case where the wavelength of light used for irradiation to acquire photoacoustic signals was a different wavelength from that scheduled for the examination, and that has been input as the imaging error cause, photoacoustic images generated from photoacoustic signals acquired by at least the same irradiation may be taken as imaging errors in a synchronized manner.

Although an example has been described in the above embodiment where an imaging error mark is displayed on or near a thumbnail image of a medical image that has been determined to be a imaging error, the present invention is not restricted to this. For example, an imaging error mark may be displayed on or near the medical image that has been determined to be a imaging error. In another example, an imaging error mark may be used that enables distinguishing between a case where a medical image has been determined to be a imaging error by user instructions, and a case where a medical image has been determined to be a imaging error synchronously with an imaging error instruction having been made regarding another medical image. In yet another example, an imaging error mark may be used that enables distinguishing of input imaging error causes.

The present invention can also be realized by processing of supplying a program that realizes one or more functions of the above-described embodiment to a system or device via a network or storage medium, and one or more processors of a computer in the system or device reading out and executing the program. The present invention can also be realized by a circuit (e.g., an application-specific integrated circuit (ASIC)) that realizes one or more functions.

The control device in the above-described embodiment may be realized as a standalone device, or may be realized in an arrangement where multiple devices are communicably combined with each other and execute the above-described processing, and both arrangements are included in an embodiment of the present invention. The above-described processing may be executed by a shared server device or server group. It is sufficient for the control device and multiple devices making up the control system to be communicable at a predetermined communication rate, and do not need to be in the same facility or within the same nation.

An embodiment of the present invention includes a form where a software program that realizes the functions of the embodiment described above is provided to a system or device, and a computer of the system or device reads out and executes code of the program that has been supplied thereto.

Accordingly, the program code that is installed to a computer to realize processing according to the embodiment by the computer is in itself an embodiment of the present invention. Also, the functions of the embodiment described above can be realized by the processing where an operating system (OS) or the like performing part or all of the actual processing, in the computer based on instructions included in the program that the computer has read out.

Forms obtained by appropriately combining the above-described embodiments are also included in an embodiment of the present invention.

The present invention is not restricted to the above-described embodiments, and various changes and modifications can be made without departing from the spirit and scope of the present invention. Accordingly, the following Claims are attached to publicly set forth the scope of the present invention.

Accordingly, an imaging error can be identified by combining a medical image regarding which there has been an imaging error instruction, and a medical image selected based on a medical image regarding which there has been an imaging error instruction, thereby improving user workflow.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A control device, comprising:
accepting means configured to accept imaging error instructions regarding a first medical image acquired based on signals of ultrasound waves from an object;
selecting means configured to select a second medical image that is a different medical image from the first medical image and that is acquired based on signals of ultrasound waves from the object, based on information attached to the first medical image; and
processing means configured to attach, to the first medical image and the selected second medical image, information indicating that the medical images are imaging errors.

2. The control device according to claim 1,
wherein the selecting means select the second medical image based on information relating to signals of ultrasound waves for acquiring the first medical image, attached to the first medical image.

3. The control device according to claim 1,
wherein the selecting means select the second medical image based on information attached to the first medical image, and information attached to each of a plurality of medical images that are different from the first medical image, and are acquired based on signals of ultrasound waves from the object, out of the plurality of medical images.

4. The control device according to claim 3,
wherein the information attached to the first medical image includes information relating to a time at which signals of ultrasound waves for generating at least the first medical image were acquired, and information attached to each of the plurality of medical images include information relating to a time at which signals of ultrasound waves for generating at least the medical images were acquired,
and wherein the selecting means select, from the plurality of medical images, a medical image generated based on signals acquired at a time within a predetermined range from the time at which the signals for generating the first medical image were acquired, as the second medical image.

5. The control device according to claim 1,
wherein, in a case where the first medical image is a photoacoustic image generated based on first photoacoustic signals that are ultrasound waves acquired by irradiating the object with light of a first wavelength,
the selecting means select a photoacoustic image that has been generated based on the first photoacoustic signals and that is a photoacoustic image different from the first medical image, as the second medical image.

6. The control device according to claim 1,
wherein, in a case where the first medical image is a photoacoustic image generated based on first photoacoustic signals that are ultrasound waves acquired by irradiating the object with light of a first wavelength, and second photoacoustic signals that are ultrasound waves acquired by irradiating the object with light of a second wavelength that is different from the first wavelength,
the selecting means select, as the second medical image, at least one of a photoacoustic image that has been generated based on the first photoacoustic signals, a photoacoustic image that has been generated based on the second photoacoustic signals, and a photoacoustic image that has been generated based on the first photoacoustic signals and the second photoacoustic signals.

7. The control device according to claim 1, further comprising:
setting means configured to set conditions for selecting the second medical image,
wherein the selecting means select the second medical image based on the settings.

8. The control device according to claim 7,
wherein the setting means set conditions for selecting the second medical image based on user instructions.

9. The control device according to claim 1, further comprising:
display control means configured to display a screen capable of input of imaging-error instructions for each of the plurality of medical images by a user, on a display unit,
wherein the selecting means select the second medical image from medical images regarding which there was no input of imaging-error instructions, out of the plurality of medical images regarding which imaging error instructions can be input on the display unit.

10. The control device according to claim 1,
wherein the processing means effect control where medical images attached with information indicating an imaging error are not transmitted to an external device, and medical images not attached with information indicating an imaging error are transmitted to an external device.

11. The control device according to claim 1,
wherein the first medical image and second medical image are each one of an ultrasound image and a photoacoustic image.

12. The control device according to claim 1,
wherein the selecting means select, as the second medical image, a medical image generated based on signals acquired after a time at which signals for generating the first medical image were acquired.

13. The control device according to claim 1,
wherein the accepting means accept imaging error cancellation instructions regarding the first medical image or the second medical image.

14. The control device according to claim 13,
wherein, in a case of the accepting means accepting an imaging error cancellation instruction regarding the first medical image,
the processing means perform imaging error cancellation by performing processing where information, attached to the first medical image and the second medical image indicating that these are imaging errors, is not attached thereto.

15. The control device according to either claim 13,
wherein the processing means effect control where imaging error-cancelled medical images can be transmitted to an external device.

16. The control device according to claim 1,
wherein the selecting means select the second medical image by using at least, out of information attached to medical images, information indicating the type of medical image, and time that the medical image was acquired.

17. A control method, comprising:
accepting imaging error instructions regarding a first medical image acquired based on signals of ultrasound waves from an object;
selecting a second medical image that is a different medical image from the first medical image and that is acquired based on signals of ultrasound waves from the object, based on information attached to the first medical image; and
processing of attaching, to the first medical image and the selected second medical image, information indicating that the medical images are imaging errors.

18. A control system, comprising:
accepting means configured to accept imaging error instructions regarding a first medical image acquired based on signals of ultrasound waves from an object;
selecting means configured to select a second medical image that is a different medical image from the first medical image and that is acquired based on signals of ultrasound waves from the object, based on information attached to the first medical image; and
processing means configured to attach, to the first medical image and the selected second medical image, information indicating that the medical images are imaging errors.

19. The control system according to claim 18, further comprising:
a probe configured to output ultrasound wave signals by transmission to and reception from an object of ultrasound waves, and output photoacoustic signals by reception of photoacoustic waves generated by irradiating the object by light; and
acquiring means configured to acquire at least one of the ultrasound wave signals and the photoacoustic signals and acquire a medical image.

20. A non-transitory computer-readable medium storing a program causing a computer to execute,
accepting imaging error instructions regarding a first medical image acquired based on signals of ultrasound waves from an object,
selecting a second medical image that is a different medical image from the first medical image and that is acquired based on signals of ultrasound waves from the object, based on information attached to the first medical image, and
processing of attaching, to the first medical image and the selected second medical image, information indicating that the medical images are imaging errors.
